SEQUENCE LISTING (32 sequences)

SEQ ID NO: 1 - DNA, 17 bp, Artificial Sequence
  PCR primer
caggaaacagctatgac

SEQ ID NO: 2 - DNA, 17 bp, Artificial Sequence
  PCR primer
ctatgcggccccattca

SEQ ID NO: 3 - DNA, 45 bp, Artificial Sequence
  PCR primer
gcgtctgcggccgcaactgtggctgcaccatctgtcttcatcttc

SEQ ID NO: 4 - DNA, 75 bp, Artificial Sequence
  PCR primer
accagccgaattcttattagcccttgtcgtcatcgtctttatagtctgagctcgcacactctcccctgttgaagc

SEQ ID NO: 5 - DNA, 82 bp, Artificial Sequence
  PCR primer
tgtgcgagctcagaacaaaaactcatctcagaagaggatctgaatggggccgcatagactgttgaaagttgtttagcaaaac

SEQ ID NO: 6 - DNA, 17 bp, Artificial Sequence
  PCR primer
gtaaaacgacggccagt

SEQ ID NO: 7 - DNA, 40 bp, Artificial Sequence
  PCR primer
gcgtctgcggccgcctccaccaagggcccatcggtcttcc

SEQ ID NO: 8 - DNA, 93 bp, Artificial Sequence
  PCR primer
gaagatctttattatgcggccccattcagatcctcttctgagatgagtttttgttctgagctcgcacaagatttgggctcaactttcttgtcc

SEQ ID NO: 9 - DNA, 62 bp, Artificial Sequence
  PCR primer
ccggaattcttattagtgatggtgatgatgatgtgagctcgcacactctcccctgttgaagc

SEQ ID NO: 10 - DNA, 33 bp, Artificial Sequence
  PCR primer
aagctcctgatctatgaggcatcccttttgcaa

SEQ ID NO: 11 - DNA, 33 bp, Artificial Sequence
  PCR primer
ttgcaaaagggatgcctcatagatcaggagctt

SEQ ID NO: 12 - DNA, 24 bp, Artificial Sequence
  PCR primer
agactctcccctgttgaagctctt

SEQ ID NO: 13 - DNA, 21 bp, Artificial Sequence
  PCR primer
ggtgctcttggaggagggtgc

SEQ ID NO: 14 - DNA, 242 bp, Artificial Sequence
  pHEN14VK Ck vector sequence
caggaaacagctatgaccatgattacgccaagcttgcatgcaaattctatttcaaggagacagtcataatgaaatacctattgcctacggcagccgctggattgttattactcgcggcccagccggccatggcgtcgacggacatccagatgacccaggcggccgcagaacaaaaactcatctcagaagaggatctgaatggggccgcatagactgttgaaagttgtttagcaaaacctcat

SEQ ID NO: 15 - protein, 47 aa, Artificial Sequence
  pHEN14VK Ck vector encoded amino acid sequence
MKYLLPTAAAGLLLLAAQPAMASTDIQMTQAAAEQKLISEEDLNGAA

SEQ ID NO: 16 - DNA, 272 bp, Artificial Sequence
  Ck vector sequence
caggaaacagctatgaccatgattacgccaagcttgcatgcaaattctatttcaaggagacagtcataatgaaatacctattgcctacggcagccgctggattgttattactcgcggcccagccggccatggccgaggtgtttgactactggggccagggaaccctggtcaccgtctcgagcggtggaggcggttcaggcggaggtggcagcggcggtggcgggtcgacggacatccagatgacccaggcggccgcagaacaaaaactcgca

SEQ ID NO: 17 - protein, 67 aa, Artificial Sequence
  Ck vector encoded amino acid sequence
MKYLLPTAAAGLLLLAAQPAMAEVFDYWGQGTLVTVSSGGGGSGGGGSGGGGSTDIQMTQAAAEQKL

SEQ ID NO: 18 - DNA, 90 bp, Artificial Sequence
  His tag/myc-tag/geneIII leader nucleic acid sequence
catcatcatcaccatcacggggccgcaatctcagaagaggatctgaatggggccgcatagactgttgaaagttgtttagcaaaacctcat

SEQ ID NO: 19 - protein, 19 aa, Artificial Sequence
  His Tag/myc-tag amino acid sequence
HHHHHHGAAISEEDLNGAA

SEQ ID NO: 20 - DNA, 221 bp, Artificial Sequence
  pACYC4VH vector nucleic acid sequence
caggaaacagctatgaccatgattacgccaagcttgcatgcaaattctatttcaaggagacagtcataatgaaatacctattgcctacggcagccgctggattgttattactcgcggcccagccggccatggccgaggtgtttgactactggggccagggaaccctggtcaccgtctcgagcgcggccgcataataaggatccagatctcatatggaattc

SEQ ID NO: 21 - protein, 41 aa, Artificial Sequence
  pACYC4VH vector encoded amnio acid sequence
MKYLLPTAAAGLLLLAAQPAMAEVFDYWGQGTLVTVSSAAA

SEQ ID NO: 22 - protein, 108 aa, Artificial Sequence
  Vk dummy amino acid sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 23 - protein, 108 aa, Artificial Sequence
  Clone C3 amino acid sequence
DIQMTQSPSSLSASVGDRVTITCRASQSISSYLNWYQQKPGKAPKLLIYAASLLQSGVPSRFSGSGSGTDFTLTISSLQPEDFATYYCQQRVYDPLTFGQGTKVEIKR

SEQ ID NO: 24 - protein, 116 aa, Artificial Sequence
  VH dummy amnino acid sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 25 - protein, 116 aa, Artificial Sequence
  Vh clone 1 amino acid sequence
EVQLLESGGGLVQPGGSLRLSCAASGFTFSSYAMSWVRQAPGKGLEWVSSIAPTGPPTTYADSVKGRFTISRDNSKNTLYLQMNSLRAEDTAVYYCAKRLTRFDYWGQGTLVTVSS

SEQ ID NO: 26 - protein, 116 aa, Artificial Sequence
  Vh clone 3 amino acid sequence
EVQLLESGGGLVQPGGSLRLSCAASGFTFSSYAMSWVRQAPGKGLEWVSSIMQGGRDTAYADSVKGRFTISRDNSKNTLYLQMNSLRAEDTAVYYCAKTRIPFDYWGQGTLVTVSS

SEQ ID NO: 27 - protein, 116 aa, Artificial Sequence
  Vh clone 4 amino acid sequence
EVQLLESGGGLVQPGGSLRLSCAASGFTFSSYAMSWVRQAPGKGLEWVSTIRDTGKNTTYADSVKGRFTISRDNSKNTLYLQMNSLRAEDTAVYYCAKMPPPFDYWGQGTLVTVSS

SEQ ID NO: 28 - protein, 116 aa, Artificial Sequence
  Vh clone 7 amino acid sequence
EVQLLESGGGLVQPGGSLRLSCAASGFTFSSYAMSWVRQAPGKGLEWVSAISTNGGMTQYADSVKGRFTISRDNSKNTLYLQMNSLRAEDTAVYYCAKIFNSFDYWGQGTLVTVSS

SEQ ID NO: 29 - protein, 116 aa, Artificial Sequence
  Vh clone 8 amino acid sequence
EVQLLESGGGLVQPGGSLRLSCAASGFTFSSYAMSWVRQAPGKGLEWVSVISTAGKQTTYADSVKGRFTISRDNSKNTLYLQMNSLRAEDTAVYYCAKTPPMFDYWGQGTLVTVSS

SEQ ID NO: 30 - protein, 116 aa, Artificial Sequence
  Vh clone 9 amino acid sequence
EVQLLESGGGLVQPGGSLRLSCAASGFTFSSYAMSWVRQAPGKGLEWVSAIQEAGPNTTYADSVKGRFTISRDNSKNTLYLQMNSLRAEDTAVYYCAKKEKVFDYWGQGTLVTVSS

SEQ ID NO: 31 - protein, 10 aa, Artificial Sequence
  GeneIII leader amino acid sequence
TVESCLAKPH

SEQ ID NO: 32 - protein, 8 aa, Artificial Sequence
  Amino acid sequence of contiguous BamHI, BglII, NdeI, and EcoRI restriction recognition sites
GSRSHMEF

(19) United States
(12) Patent Application Publication
(10) Pub. No.: US 2006/0280734 A1
(43) Pub. Date: Dec. 14, 2006

(54) Retargeting

(75) Inventors: Gregory Paul Winter, Cambridge (GB); Ian Michael Tomlinson, Cambridge (GB); Olga Ignatovich, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Domantis Limited

(21) Appl. No.: 11/165,672

(22) Filed: Jun. 24, 2005

Related U.S. Application Data
(63) Continuation of application No. PCT/GB03/05657, filed on Dec. 24, 2003.

(30) Foreign Application Priority Data
Dec. 27, 2002 (GB) 0230201.6

(51) Int. Cl. A61K 39/395; C07K 16/44; C07H 21/04; C12P 21/06; C12N 5/06

(52) U.S. Cl. 424/133.1; 435/69.1; 435/320.1; 435/327; 530/387.3; 536/23.53

(57) ABSTRACT

The present invention relates to a method for the generation of immunoglobulin molecules of predetermined specificity. In particular, the invention relates to a method for the retargeting of the epitope binding specificity of one or more antibodies using single variable domains which exhibit a dominant epitope binding specificity.

The present invention relates to a method for the modulation of the epitope binding specificity of immunoglobulin molecules. In particular, the invention relates to the retargeting of the epitope binding specificity of one or more antibodies using single variable domains of defined specificity.

### INTRODUCTION

The antigen binding domain of an antibody comprises two separate regions: a heavy chain variable domain (V_{H}) and a light chain variable domain (V_{L}: which can be either V_{Kappa} or V_{Lamda}). The antigen binding site itself is formed by six polypeptide loops: three from V_{H} domain (H1, H2 and H3) and three from V_{L} domain (L1, L2 and L3). A diverse primary repertoire of V genes that encode the V_{H} and V_{L} domains is produced by the combinatorial rearrangement of gene segments. The V_{H} gene is produced by the recombination of three gene segments, V_{H}, D and J_{H}. In humans, there are approximately 51 functional V_{H} segments (Cook and Tomlinson (1995) Immunol Today, 16: 237), 25 functional D segments (Corbett et al. (1997) J. Mol. Biol., 268: 69) and 6 functional J_{H} segments (Ravetch et al. (1981) Cell, 27: 583), depending on the haplotype. The V_{H} segment encodes the region of the polypeptide chain which forms the first and second antigen binding loops of the V_{H} domain (H1 and H2), whilst the V_{H}, D and J_{H} segments combine to form the third antigen binding loop of the V_{H} domain (H3). The V_{L} gene is produced by the recombination of only two gene segments, V_{L} and J_{L}. In humans, there are approximately 40 functional V_{κ} segments (Schäble and Zachau (1993) Biol. Chem. Hoppe-Seyler, 374: 1001), 31 functional V_{L} segments (Williams et al. (1996) J. Mol. Biol., 264: 220; Kawasaki et al. (1997) Genome Res., 7: 250), 5 functional J_{κ} segments (Hieter et al. (1982) J. Biol. Chem., 257: 1516) and 4 functional J_{λ} segments (Vasicek and Leder (1990) J. Exp. Med., 172: 609), depending on the haplotype. The V_{L} segment encodes the region of the polypeptide chain which forms the first and second antigen binding loops of the V_{L} domain (L1 and L2), whilst the V_{L} and J_{L} segments combine to form the third antigen binding loop of the V_{L} domain (L3). Antibodies selected from this primary repertoire are believed to be sufficiently diverse to bind almost all antigens with at least moderate affinity. High affinity antibodies are produced by “affinity maturation” of the rearranged genes, m which point mutations are generated and selected by the immune system on the basis of improved binding.

Antibodies bind their antigen via a complementary V_{H}/V_{L} pair comprising a complementary binding site. There is some evidence that in certain circumstances two different antibody binding specifities might be incorporated into the same binding site. For example, cross-reactive antibodies have been described, usually where the two antigens are related in sequence and structure, such as hen egg white lysozyme and turkey lysozyme (McCafferty et al., WO 92/01047) or to free hapten and to hapten conjugated to carrier (Griffiths AD et al., EMBO J 1994 13:14 3245-60. Furthermore natural autoantibodies have been described that are polyreactive (Casali & Notkins, Ann. Rev. Immunol. 7, 515-531), reacting with at least two (usually more) different antigens that are not structurally related. It has also been shown that selections of random peptide repertoires using phage display technology on a monoclonal antibody will identify a range of peptide sequences that fit the antigen binding site. Some of the sequences are highly related, fitting a consensus sequence, whereas others are very different and have been termed mimotopes (Lane & Stephen, Current Opinion in Immunology, 1993, 5, 268-271). It is therefore clear that the antigen binding site of an antibody, comprising associated and complementary V_{H} and V_{L} domains, has the potential to bind to many different antigens from a large universe of known antigens. It is less clear how to create a binding site to two given antigens, particularly those which are not necessarily structurally related. Most recently, ‘dual-specific’ immunoglobulin molecules have been described. Detailed are provided in WO03/002609 in the name of the present inventors.

Single heavy chain variable domains have been described, derived from natural antibodies which are normally associated with light chains (from monoclonal antibodies or from repertoires of domains; see EP-A-0368684). These domains have been shown to bind to their antigen specifically.

However, at the filing date of the present invention, it has not been reported that single light chain domains are able to bind to their antigen specifically.

### SUMMARY OF THE INVENTION

The present inventors have surprisingly shown that single light chain variable domains of a defined epitope specificity can be selected and incorporated into an antibody molecule and confer upon that molecule the epitope specificity of the single light chain variable domain.

Furthermore, the inventors have surprisingly found that an antibody molecule of desired epitope specificity may be generated by selecting one or more light chain variable domain/s and replacing one or more light chain variable domains of an intact antibody molecule with the one or more selected domain/s to create a retargeted antibody The present inventors have therefore surprisingly shown that in some instances despite the presence of heavy chain variable domains within the retargeted antibodies antigen binding site, the specificity of epitope binding conferred by the selected light chain domains predominates.

Importantly, the present inventors have shown that the methods of the invention may be used to retarget the epitope binding specificity of an antibody molecule as herein described such that the resultant retargeted antibody molecule is capable of epitope binding to an epitope which is not structurally related to the specific epitope of the same antibody molecule prior to the retargeting process.

The inventors consider that the method may be used for retargeting of antibodies to ‘non-structurally related epitope binding specificities’ due to the finding that in certain circumstances the epitope binding specificity of the light chain dominates, and in these circumstances, when the light chain variable domain of an epitope binding site is replaced by a second variable domain which exhibits a different dominant epitope binding specificity, then the binding specificity of the second ‘dominant epitope binding specificity’ predominates.

This discovery and method for the generation of such antibodies is in contrast to those methods described for the generation of cross-reactive antibodies such as those described in McCafferty et al;, WO92/01047. Such antibodies generated using the methods described in McCafferty et al, bind to more than one epitope (that is are cross-reactive), and their epitope binding specificity is retargeted only to the extent that the antibodies generated are able to bind a first epitope and also a second structurally related epitope (thus there is no switching of epitope binding specificity from one epitope to another, there is a mere addition of epitope binding specificity). Moreover such methods cannot be used to generate antibodies which can be retargeted to bind a first and a second non-structurally related epitope. The inventors consider that this important difference arises from the nature of epitope binding in these cross-reactive antibodies where both the light chain and the heavy chain variable domains participate in determining the epitope binding specificity. That is, unlike in the methods of one aspect of the present invention, the binding specificity of the light chain variable domain does not predominate.

Thus, in a first aspect, the present invention provides a method for generating an immunoglobulin molecule of predetermined epitope binding specificity comprising the steps of:
(a) selecting an antibody light chain variable domain comprising an epitope binding specificity, and
(b) operably linking the antibody single light chain variable domain to an immunoglobulin skeleton.

According to the above aspect of the invention preferably, more than one light chain variable domain is selected according to step (a) and linked to an immunoglobulin skeleton according to step (b), wherein each domain comprises an epitope binding specificity.

According to the present invention ‘an immunoglobulin molecule’ refers to a family of polypeptides which retain the immunoglobulin fold characteristic of antibody molecules, which contains two β sheets and, usually, a conserved disulphide bond. Members of the immunoglobulin superfamily are involved in many aspects of cellular and noncellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor).

According to the above aspect of the invention, preferably the immunoglobulin molecule is an antibody molecule as herein defined. More advantageously, the antibody molecule is scFv or a Fab′ or an IgG. Most advantageously, the antibody may be ‘dual-specific’ as herein defined.

According to the present invention an ‘epitope’ is a unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

As used herein the term ‘predetermined epitope binding specificity’ means a defined or known epitope binding specificity. Likewise, the term the ‘epitope binding specificity’ denotes which epitope/s are specifically bound by the single light chain variable domains and or heavy chain variable domains according to the present invention Antigens may comprise one or more epitopes. In the examples herein, the specific antigen is BSA (bovine serum albumin).

According to the present invention, the term ‘selecting’ means choosing from a number of different alternatives. Those skilled in that art will be aware of methods of selecting one or more antibody variable domains according to the present invention. Advantageously, the method involves selecting from a library. Advantageously, the library is a phage display library.

An antigen ‘single light chain variable domain’ as referred to herein describes a single polypeptide domain which, in its native environment, is part of an antibody light chain and along with a heavy chain variable domain forms an antigen binding site of an immunoglobulin molecule, as well as struturally similar molecules which may be synthetic in origin. Specifically, the binding site is the site formed by the hypervariable loops of variable domain/s which interact specifically with an epitope as herein defined.

For the avoidance of any doubt, the term ‘single’ (light chain variable domain) means that the light chain variable domain is free from at least any heavy chain variable domain.

The present inventors have found that variable domains may be of a dominant epitope binding specificity or a co-domiant epitope binding specificity. The term ‘a dominant light chain epitope binding specificity’ means that in the event that the light chain variable domain of an antibody comprising a complementary V_{H}/V_{L} epitope binding site is replaced by a light chain variable domain comprising a ‘dominant epitope binding specificity’, then the resultant complementary V_{H}/V_{L} epitope binding site will adopt the epitope binding specificity of the V_{L} domain. The inventors have also shown that dominance may also be displayed for VL/VL combinations and certain VH/VH combinations as described herein.

Advantageously according to the above aspect of the invention, advantageously, the variable light chain variable domain is of a ‘dominant epitope binding specificity’.

According to the present invention, the term ‘immunoglobulin skeleton’ refers to a protein which comprises at least one immunoglobulin fold and which acts as a scaffold for linking to one or more antibody single light chain variable domains, as defined herein. Preferably the ‘immunoglobulin skeleton’ as herein defined comprises one or more antibody heavy chain variable domain/s such that together one or more antibody heavy chain variable domain/s and one or more selected antibody single light chain variable domains as referred to in step (a) form an antigen binding site.

In a preferred embodiment of the above aspect of the present invention, ‘an immunoglobulin skeleton’ is an complete antibody molecule of the IgG subclass lacking at least one light chain variable domain.

Further preferred immunoglobulin skeletons as herein defined include any one or more of those selected from the following: an immunoglobulin molecule comprising (i) the CL (kappa or lambda subclass) domain of an antibody; (ii) at least the CH1 domain of an antibody heavy chain; an immunoglobulin molecule comprising the CH1 and CH2 domains of an antibody heavy chain; an imnunoglobulin molecule comprising the CH1, CH2 and CH3 domains of an antibody heavy chain; or any of the above subset (ii) in conjunction with the CL (kappa or lambda subclass) domain of an antibody. Those skilled in the art will be aware that this list is not intended to be exhaustive and will be aware of other suitable immunoglobulin skeletons.

As referred to herein the term ‘virgin antibody heavy chain domains’ refers to antibody heavy chain domains which have not been through the processes of in vitro selection with one or more antigen/s so as to select those antibody heavy chain domains which can bind to one or more antigens specifically.

As referred to herein the term ‘operably linking’ (the one or more antibody single light chain variable domains to an immunoglobulin skeleton) means that one or more antibody light chain variable domains are linked to an immunoglobulin skeleton as herein defined in such a way that the one or more antibody light chain variable domains are capable of binding to one or more antigens specifically. That is, the term “operably linked” refers to a juxtaposition wherein the components described are in a relationship permitting them to function in their intended manner. As referred to herein the term ‘linking’ includes within its scope the use of a linker sequence. Methods of iking the one or more antibody single light chain variable domains to an immunoglobulin skeleton include the use of ‘linking sequences’ which may be polypeptide linkers, or synthetic linkers. It also includes within its scope the direct attachment of the immunoglobulin scaffold to one or more antibody light chain variable domains.

Linking of the skeleton to the one or more single light chain variable domains, as herein defined may be achieved at the polypeptide level, that is after expression of the nucleic acid encoding the skeleton and/or the single light chain domain. Alternatively, the linking step may be performed at the nucleic acid level. Methods of linking an immunoglobulin skeleton according to the present invention, to the one or more light chain variable domains include the use of protein chemistry and/or molecular biology techniques which will be familiar to those skilled in the art and are described herein.

For the avoidance of doubt an ‘epitope binding site’ as referred to herein describes the combination of one or more heavy chain variable domains and one or more complementary light chain variable domains which together forms a binding site which permits the specific binding of one or more epitopes to the immunoglobulin molecule.

According to the aspects of the invention recited above, an immunoglobulin molecule generated adopts the epitope binding specificity of the antibody single light chain variable domain.

In a further aspect, the present invention provides a method for retargeting the epitope binding specificity of an antibody molecule such that the resultant antibody molecule is not cross-reactive with the epitope capable of being specifically bound by the antibody molecule prior to retargeting which method comprises the steps of:
(a) selecting an antibody variable domain comprising an epitope binding specificity and
(b) replacing the light chain variable domain/s or the heavy chain variable domains of an epitope binding site comprised by the antibody molecule to be retargeted with the antibody variable domain selected according to step (a), wherein the selected variable domain comprises at least one dominant epitope binding specificity.

According to the above aspect of the invention, preferably the variable domain/s replaced according to step (b) above are light chain variable domain/s. More advantageosuly, a light chain variable domain is selected according to step (a) above and used to replace a light chain variable domain according to step (b) above.

In an alternative embodiment of the above aspect of the invention, the variable domain/s replaced according to step (b) above are heavy chain variable domain/s. More advantageosuly, a heavy chain variable domain is selected according to step (a) above and used to replace a heavy chain variable domain according to step (b) above.

According to the above aspect of the invention, advantageously, the antibody variable domain selected according to step (a) comprises an epitope binding specificity wherein the epitope is not structurally related to the epitope specifically bound by the antibody prior to retargeting according to the method of the above aspect of the invention.

According to the methods of the invention, ‘non-structurally related epitopes’ may be present on the same antigen or on different non-structurally antigens. Advantageously, they will be present on different/non-structurally related antigens.

As referred to herein the term ‘structurally related’ (epitopes) means in its broadest sense that in the case where there exists a first and a second ‘structurally related’ epitope then a monoclonal antibody may be generated which is raised to the first or second epitope but which is capable of specifically binding to both epitopes. This phenomenon ocurrs because the two epitopes are sufficiently structurally similar that the monoclonal antibody cannot distinguish (in terms of specific binding) between the two epitopes. Thus as described herein, in the case where two epitopes are not structurally related then monoclonal antibodies raised to one of the epitopes do not exhibit the phenomemon of cross-reactivity.

Structurally unrelated epitopes and/or antigens may also or alternatively be identified on the basis of sequence identity at the amino acid level between the respective epitopes and/or antigens. Such information may be presented as a % identity of amino acid sequence between the two respective epitopes and/or antigens. Thus as referred to herein the term ‘structurally unrelated epitopes’ and ‘structurally unrelated antigens’ includes within its scope an amino acid sequence identity between the two respective epitopes or antigens respectively of 30% or less, preferably 29%, 28%, 27%, 26% or less. Most advantageously, ‘structurally unrelated epitopes’ and/or ‘structurally unrelated antigens’ share a amino acid sequence identity of 25% or less.

Methods for the determination of the % identity between two or more respective amino acid sequences will be familiar to those skilled in the art and are described herein.

In a further preferred embodiment of the above aspect of the invention, advantageously the method may comprise a further step which removes from those antibodies generated contaminant cross-reactive antibodies and wherein step (c) comprises testing those retargeted antibodies generated using the method of step (a) and step (b) for their ability to cross-react with the epitope or antigen capable of being specifically bound by the antibody molecule prior to retargeting, and removing those antibodies which exhibit that property from those antibodies retargeted according to step (a) and step (b) described above.

For the avoidance of any doubt, according to the above aspect of the invention, ‘non-structurally related epitopes’ may be present on the same antigen or on different antigens. Advantageously, they will be present on different (non-structurally related) antigens.

According to the above aspect of the invention preferably more than one epitope binding specificity is retargeted.

Preferably, the selected variable domain is a heavy chain variable domain. Alternatively, the selected variable domain is a light chain variable domain.

According to the present invention, the term ‘a dominant epitope binding specificity’ means that in the event that the light chain variable domain of an antibody comprising a complementary V_{H}/V_{L} epitope binding site is replaced by a light chain variable domain comprising a ‘dominant epitope binding specificity’, then the resultant complementary V_{H}/V_{L} epitope binding site will adopt the epitope binding specificity of the V_{L} domain.

Accordingly, in the event that the heavy chain variable domain of an antibody comprising a complementary V_{H}/V_{L} epitope binding site is replaced by a heavy chain variable domain comprising a ‘dominant epitope binding specificity’, then the resultant complementary V_{H}/V_{L} epitope binding site will adopt the epitope binding specificity of the V_{H} domain.

Accordingly, a ‘co-dominant light chain epitope binding specificity’ means that in the event that the light chain variable domain of an antibody comprising a complementary V/V_{L} epitope binding site is replaced by a light chain variable domain comprising a ‘co-dominant light chain epitope binding specificity’ then the resultant complementary V_{H}/V_{L} epitope will comprise two epitope binding specificities, one provided by the V_{H} domain and one provided by the V_{L} domain. That is the epitope binding specificity of neither the V_{L} domain or the V_{H} domain predominates. Such an antibody is referred to as a ‘Dual-Specific Antibody’. Such molecules are described in detail in WO03/002609in the name of the present inventors which is incorporated herein by reference. Those skilled in the art will appreciate that the use of co-dominant epitopes may be used to generate V_{L}/V_{L} dual-specifics and also V_{H}/V_{H} dual-specifics. It is important to note that the use of co-dominant epitopes may be used for the generation of ‘dual-specific’ antibodies as herein described wherein the two epitopes capable of specific binding by the dual-specific antibody do not have to be structurally related. According to the methods of the present invention, preferably the two epitopes are not structurally related.

The inventors realised that the methods of the present invention could be used to generate antibodies having more than one epitope binding specificity.

Thus in a further aspect the present invention provides a method for the generation of an antibody of more than one epitope binding specificity comprising the steps of:
(a) selecting an antibody variable domain comprising one or more epitope binding specificities; and
(b) replacing the light or the heavy chain variable domain of at least one epitope binding site but not all of the epitope binding sites comprised by the antibody molecule, with the antibody variable domain selected according-to step (a).

According to the above aspect of the invention, advantagously the light chain variable domain is replaced according to step (b) above.

Advantageously, more than one epitope binding site is retargeted.

The selected variable domain is preferably a heavy chain variable domain; alternatively, it is a light chain variable domain.

According to the above aspect of the invention, the light chain variable domain may be kappa or lambda. Advantageously, the light chain variable domain is selected from the kappa family of light chains.

According to the present invention, the term ‘antibody’ or ‘antibody molecule’ includes within its scope IgG, IgM, IgA, IgD or IgE and fragments such as a Fab, F(ab′)₂, Fv, disulphide linked Fv, scFv, diabody, dual-specific antibodies whether derived from any species naturally producing an antibody, or created by recombinant DNA technology; whether isolated from serum, B-cells, hybridomas, transfectomas, yeast or bacteria.

In addition, the term ‘antibody’ or ‘antibody molecule’ includes within its scope any fragment of an antibody molecule as herein defined as long as that fragment comprises at least one antigen binding site comprising at least one light chain variable domain and at least one heavy chain variable domain wherein the antigen binding site permits the specific binding of one or more antigens to one or more antibodies of the present invention.

As herein defined, the term ‘retargeting the epitope binding specificity’ includes within its scope altering/changing the epitope binding specificity of the antibody molecule from that exhibited in the native/untargeted antibody (comprising at least one epitope binding site and described above) to that exhibited by a variable domain selected according to the present invention. Advantageously, it involves altering/changing the epitope binding specificity to that exhibited by a light chain variable domain. Alternatively in certain circumstances it may involve using a selected V_{H} to retarget V_{H}/V_{H} multispecific ligands. Those skilled in the art will appreciate that any given antibody molecule may possess more than one epitope binding specificity. As such only one of the epitope binding specificities of an antibody molecule may be ‘retargeted’ or more than one of the specificities may be ‘retargeted’ according to the methods of the present invention. In addition one skilled in the art will appreciate that an antibody molecule may comprise more than one antigen binding site which may be of the same or of differing epitope specificities. In the case where there is more than one antigen binding site sharing a common epitope binding specificity, then only one of the epitope binding sites may be retargeted or more than one may be retargeted according to the methods described herein. Advantageously, all of the antigen binding sites and/or epitope binding specificities of an antibody molecule are retargeted as herein defined.

As referred to herein an ‘epitope binding site’ describes the combination of one or more heavy chain variable domains and one or more light chain variable domains which are complementary to one another and together forms a binding site which permits the specific binding of antigen to the antibody molecule comprising it.

According to the above aspect of the invention, the term ‘replacing’ (the light chain variable domain/s of at least one epitope binding site with at least one antibody light chain variable domain) means substituting (the variable light chain comprising the epitope binding site) with at least one light chain variable domain according to the present invention.

In a further aspect, the present invention provides an immunoglobulin molecule of predetermined epitope binding specificity obtainable using a method of the present invention.

Advantageously, the immunoglobulin molecule is an antibody molecule. Most advantageously, the antibody molecule is a full IgG, an scFv or a Fab.

In a further aspect still the present invention provides a retargeted antibody molecule obtainable using the methods of the invention.

According to the above aspect of the invention, the light chain variable domain is selected from the kappa or lambda family of light chains. Advantageously, the light chain variable domain used to retarget immunoglobulins in accordance with the present invention comprises a ‘dominant’ epitope binding specificity.

According to the present invention, the term ‘a dominant light chain epitope binding specificity’ describes the situation that in the event that the light chain variable domain of an antibody comprising a complementary V_{H}/V_{L} epitope binding site is replaced by a light chain variable domain comprising a ‘dominant epitope binding specificity’, then the resultant complementary V_{H}/V_{L} epitope binding site will adopt the epitope binding specificity of the V_{L} domain. That is, the epitope binding specificity of the light chain variable domain predominates.

In a further aspect still, the present invention provides a nucleic acid construct encoding an immunoglobulin molecule of predetermined epitope binding specificity or a nucleic acid construct encoding a retargeted antibody molecule as herein described.

In yet a further aspect, the present invention provides a vector comprising a nucleic acid construct according to the present invention

Advantageously, the vector further comprises components necessary for the expression of an immunoglobulin molecule of predetermined antigen binding specificity or a retargeted antibody molecule according to the present invention.

In a further aspect still, the present invention provides a host cell transfected with a nucleic acid construct or a vector according to the present invention.

In a further aspect still, the present invention provides a diagnostic and/or assay kit comprising at least an immunoglobulin molecule or an antibody molecule according to the present invention.

In addition to the finding reported above, the present inventors have also found that the V_{L}

or V_{H} domain used to retarget doesn't always predominate. For example, in the case that a light chain variable domain is used to retarget an antibody comprising a complementary VH/VL epitope binding site, in the event that the V_{L} domain doesn't predominate, then the resultant antibody molecule will comprise a V_{L} domain and a V_{H} domain which together from a complementary epitope binding site with two epitope binding specificities, one provided by the V_{H} domain and the other provided by the V_{L} domain. Such antibodies are known as ‘Dual-specific antibodies’ and are described in WO03/002609, which was filed by the present inventors and which is herein incorporated by reference.

Thus in a further aspect the present invention provides a method for the generation of a dual-specific antibody comprising the steps of:
(a) selecting an antibody single variable chain domain comprising an epitope binding specificity, wherein at least one epitope binding specificity of the variable domain is co-dominant; and
(b) Replacing the light chain variable domain or the heavy chain variable domain of at an epitope binding site comprising the antibody molecule with the antibody variable chain domain selected according to step (a), such that each variable domain of the epitope binding site binds a respective epitope wherein the respective epitopes are not identical to one another

According to the above aspect of the invention, the respective epitopes bound by each variable domain of each epitope binding site according to step (b) are not identical, such that an antibody molecule is created which comprises more than one epitope binding specificity.

As referred to herein, the term ‘dual-specific antibody’ means an antibody molecule which comprises at least one complementary epitope binding site comprising one light chain variable domain and one heavy chain domain, wherein each variable domain possesses an independent binding specificity. That is, the heavy chain variable domain comprises one epitope binding specificity and the light chain variable domain comprises a further epitope binding specificity such that a dual-specific antibody may possess dual-epitope specificity. In addition it also includes within its scope dual-specific VH/VH combinations and VL/VL combinations.

It is an important feature of the above aspect of the invention that the VH and VL domains comprising the complementary epitope binding site of a ‘dual-specific antibody’ generated according to the method of the invention do not cooperate in binding to:specific epitopes unlike the cross-reactive antibodies generated using the method described in McCafferty et al., WO 92/01047.

Thus according to the above aspect of the invention, it is not a requirement of the method that the two epitopes capable of being specifically bound by the dual-specific antibody are structurally related. Advantageously, the two epitopes bound by the epitopes capable of being bound by the dual-specific antibody generated according to the method of the invention are not structurally related.

According to the above aspect of the invention, ‘non-structurally related epitopes’ may be present on the same antigen or on different antigens. Advantageously, they will be present on different antigens.

According to the above aspect of the invention, the variable domains may be light chain variable domains or heavy chain variable domains. Advantageously, they are light chain variable domains.

As described herein a ‘co-dominant epitope binding specificity’ means that in the event that the light chain variable domain of an antibody comprising a complementary V_{H}/V_{L} epitope binding site is replaced by a light chain variable domain comprising a ‘co-dominant epitope binding specificity’ then the resultant complementary V_{H}/V_{L} epitope will comprise two epitope binding specificities, one provided by the V_{H} domain and one provided by the V_{L} domain. That is the epitope binding specificity of neither the V_{L} domain or the V_{H} domain predominates. Such an antibody is referred to as a ‘Dual-Specific Antibody’.

According to the above aspect of the invention, preferably the dual-specific antibody is generated by replacing the light chain variable domain in step (b) referred to above. Advantageously, the light chain variable domain of an IgG molecule is replaced according to step (b) of the above aspect of the invention. More advantageously, the light chain variable domain according to step (b) of the method referred to above is replaced by a second light chain variable domain comprising an second different epitope binding specificity.

According to the above aspect of the invention, preferably the dual-specific antibody molecule generated has the format of an IgG molecule.

In an alternative preferred embodiment of the above aspect of the invention, a dual-specific antibody is generated by replacing the heavy chain variable domain referred to in step (b) above with a second variable domain which comprises a second/alternative epitipe binding specificity. Advantageously, the second variable domain is a heavy chain variable domain. More preferably, according to the above aspect of the invention, an IgG molecule is created by replacing the heavy chain variable domain of that IgG molecules epitope binding site. Most preferably, the heavy chain variable domain of an IgG molecule is replaced by a heavy chain variable domain comprising a second/alternative epitope binding specificity.

The present inventors also realised that the methods of the present invention may be used for the retargeting of polyclonal antibodies. Significantly, polyclonal antisera often comprise epitope specificities which react to one or more human ‘self-epitopes’. This limits the therapeutic potential of such antisera. By retargeting the majority, or advantageously all ‘self epitopes’, then polyclonal antisera may be of great therapeutic use. For example retargeted polyclonal antisera may be of use in targeting cancer cells and/or other diseased cells.

Thus in a further aspect the present invention provides a method for retargeting the epitope binding specificity of a polyclonal antiserum comprising the step of:
(a) Selecting an antibody variable domain comprising an epitope binding specificity wherein the variable domain comprises a dominant epitope binding specificity; and
(b) Replacing the light chain variable domain or the heavy chain variable domain of at least a proportion of those antibodies comprising the antiserum with the antibody variable chain domain selected according to step (a).

According to the above aspect of the invention, the one or more variable chain domains may be light chain variable domains or heavy chain variable domains. Advantageously they are light chain variable domains.

According to the above aspect of the invention, preferably at least 1% of all those epitope binding specificities comprised within the antiserum are retargeted as defined herein. More preferably at least 2%, 3%, 4%, 5%, 6%, 7%, 8%, 9%, 10% of all of those epitope binding specificities comprising the antibodies binding site are retargeted as herein defined. More preferably still at least, 11%, 12%, 13%, 14%, 15%, 16%, 17%, 18%, 19% 20% of all those epitope binding specificities comprising the antiserum are retargeted as herein defined. Most preferably 30%, 40%, 50%, 60%, 70%, 80%, 90%, 95% of all those epitope binding specificities as herein defined comprised within the polyclonal antiserum are retargeted. Most advantageously, 100% or substantially 100% of those epitope binding specificities comprised within the antiserum are retargeted.

According to the above aspect of the invention, advantageously, those epitope binding specificities retargeted are a ‘self-epitope binding specificity’. By the term ‘self-epitope binding specificity’ (of an antibody molecule) it is meant that the binding specificity of the antibody is that of a self epitope, that is an epitope which is commonly found in a human or animal body, advantageously a human body.

The inventors have shown that practically a particularly effective method to retarget a population of monoclonal or polyclonal antibodies (which may be isolated from a patient or generated in vitro in cell culture) is to retarget one or more of the antibodies by replacing the endogenous VL-CL fragment whereby the VH has a given binding specificity X to produce a new produce a new population of antibodies with the given binding specificity X. Alternatively one or more of the antibodies may be retargetted by replacing the endogenous VH-CL fragment whereby the VH has a given binding specificity X to produce anew population of antibodies with a given binding specificity X. In an alternative embodiment the VL-CL or VH-CL could be dropped into a suitable IgG expression vector inself to create the same product.

### BRIEF DESCRIPTION OF THE FIGURES

FIG. 1 shows a pHEN14Vₖ vector

FIG. 2 shows a C_{κ}vector as herein described

FIG. 3 shows the vector pIT2.

FIG. 4 shows a Cₖ/gIII phagemid

FIG. 5 shows a PACYCV_{H} vector

FIG. 6 shows a C_{H} vector

FIG. 7 shows the C3 clone chosen for further analysis.

FIG. 8 shows the specific binding of Cₖ/Cₖ clone C3 to BSA.

FIG. 9 shows that there is no cross-reactivity in binding of Cₖ/Cₖ clone C3 binding.

FIG. 10 shows isolated the VH chain sequences of isolated clones.

FIG. 11 shows an IgG molecule specfic for BSA

FIG. 12 shows the results of ELISA experiments which demonstrate the presence of IgG molecules which can bind BSA specifically.

### DETAILED DESCRIPTION OF THE INVENTION

Definitions

Immunoglobulin This refers to a family of polypeptides which retain the immunoglobulin fold characteristic of antibody molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). Preferably, the present invention relates to antibodies.

Domain A domain is a folded protein structure which retains its tertiary structure independently of the rest of the protein. Generally, domains are responsible for discrete functional properties of proteins, and in many cases may be added, removed or transferred to other proteins without loss of function. By single antibody variable domain we mean a folded polypeptide domain comprising sequences characteristic of antibody variable domains. It therefore includes antibody variable domains, for example in which one or more loops have been replaced by sequences which are not characteristic of antibody variable domains, or antibody variable domains which have been truncated or comprise N- or C-terminal extensions.

Repertoire A collection of diverse variants, for example polypeptide variants which differ in their primary sequence. A library used in the present invention will encompass a repertoire of polypeptides comprising at least 1000 members.

Library The term library refers to a mixture of heterogeneous polypeptides or nucleic acids. The library is composed of members, which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

Antibody An antibody (for example IgG, IgM, IgA, Igd or IgE) or fragment (such as a FAb, F(Ab′)₂, Fv, disulphide linked Fv, scFv, diabody, dual-specific antibody) whether derived from any species naturally producing an antibody, or created by recombinant DNA technology; whether isolated from serum, B-cells, hybridomas, transfectomas, yeast or bacteria).

As referred to herein, the term ‘dual-specific antibody’ means an antibody molecule which comprises at least one complementary epitope binding site comprising one light chain variable domain and one heavy chain domain, wherein each variable domain posesses an independent binding specificity. That is, the heavy chain variable domain comprises one epitope binding specificity and the light chain variable domain comprises a further epitope binding specificity such that a dual-specific antibody may possess dual-epitope specificity and wherein the light chain variable domain and heavy chain variable domain do not cooperate in epitope binding. In addition it also includes within its scope dual-specific VH/VH combinations and VL/VL combinations. Advantageously, a dual-specific ligand generated according to the method of the invention is capable of binding to two non-structurally related epitopes.

Epitope A unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

‘Epitope binding specificity’ denotes which epitope/s are specifically bound by the single light chain variable domains and or heavy chain variable domains according to the present invention. Antigens may comprise one or more epitopes. In the examples herein, the specific antigen is BSA (bovine serum albumin).

The term retargeting the epitope binding specificity includes within its scope altering/changing the epitope binding specificity of the antibody molecule from that exhibited in the native/untargeted antibody (comprising at least one epitope binding site and described above) to that exhibited by a single variable chain domain selected according to the present invention. Advantageously, the epitope binding specificity is altered to that exhibited by a single light chain variable domain. Those skilled in the art will appreciate that any given antibody molecule may possess more than one epitope binding specificity. As such only one of the epitope binding specificities of an antibody molecule may be ‘retargeted’ or more than one of the specificities may be ‘retargeted’ according to the methods of the present invention. In addition one skilled in the art will appreciate that an antibody molecule may comprise more than one epitope binding site which may be of the same or of differing epitope binding specificity. In the case where there is more than one epitope binding site sharing a common epitope binding specificity, then only one of the epitope binding sites may be retargeted or more than one may be retargeted according to the methods described herein. Advantageously, all of the epitope binding sites and/or epitope binding specificities of an antibody molecule are retargeted as herein defined

Antigen A ligand that binds to a small fraction of the members of a repertoire as herein defined. Advantageously, an antigen will bind specifically to one or more members of a repertoire and is known as a specific antigen. A specific antigen comprises one or more epitopes. It may be a polypeptide, protein, nucleic acid or other molecule.

The term selecting means choosing from a number of different alternatives. Those skilled in that art will be aware of methods of selecting one or more antibody single light chain domains. Advantageously, the method involves selecting from a library. Advantageously, the library is a phage display library.

Universal framework A single antibody framework sequence corresponding to the regions of an antibody conserved in sequence as defined by Kabat (“Sequences of Proteins of Immunological Interest”, US Department of Health and Human Services) or structure as defined by Chothia and Lesk, (1987) J. Mol. Biol. 196:910-917. The invention provides for the use of a single framework, or a set of such frameworks, which has been found to permit the derivation of virtually any binding specificity though variation in the hypervariable regions alone.

General Techniques

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

Preparation of Antibodies

Antibodies according to the invention may be prepared according to previously established techniques, used in the field of antibody engineering, for the preparation of scFv, “phage” antibodies and other engineered antibody molecules. Techniques for the preparation of antibodies are for example described in the following reviews and the references cited therein: Winter & Milstein, (1991) Nature 349:293-299; Plueckthun (1992) Immunological Reviews 130:151-188; Wright et al., (1992) Crti. Rev. Immunol.12:125-168; Holliger, P. & Winter, G. (1993) Curr. Op. Biotechn. 4, 446-449; Carter, et al. (1995) J. Hematother. 4, 463-470; Chester, K. A. & Hawkins, R. E. (1995) Trends Biotechn. 13, 294-300; Hoogenboom, H. R. (1997) Nature Biotecbnol. 15, 125-126; Fearon, D. (1997) Nature Biotechnol. 15, 618-619; Plückthun, A. & Pack, P. (1997) Immunotechnology 3, 83-105; Carter, P. & Merchant, A. M. (1997) Curr. Opin. Biotechnol. 8, 449-454; Holliger, P. & Winter, G. (1997) Cancer Immunol. Immunother. 45,128-130.

The techniques employed for selection of the variable domains employ libraries and selection procedures which are known in the art. Natural libraries (Marks et al. (1991) J. Mol. Biol., 222: 581; Vaughan et al. (1996) Nature Biotech., 14: 309) which use rearranged V genes harvested from human B cells are well known to those skilled in the art. Synthetic libraries (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97) are prepared by cloning immunoglobulin V genes, usually using PCR Errors in the PCR process can lead to a high degree of randomisation. V_{L} libraries may be selected against target antigens, in which case single domain binding is directly selected for.

A. Library Vector Systems

A variety of selection systems are known in the art which are suitable for use in the present invention. Examples of such systems are described below.

Bacteriophage lambda expression systems may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. Whilst such expression systems can be used to screening up to 10⁶ different members of a library, they are not really suited to screening of larger numbers (greater than 10⁶ members).

Of particular use in the construction of libraries are selection display systems, which enable a nucleic acid to be linked to the polypeptide it expresses. As used herein, a selection display system is a system that permits the selection, by suitable display means, of the individual members of the library by binding the generic and/or target ligands.

Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encoding them) for the in vitro selection and amplification of specific antibody fragments that bind a target antigen (McCafferty et al., WO 92/01047). The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) Nature, 348: 552; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci U.S.A., 88:10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lemer et al. (1992) Science, 258: 1313, incorporated herein by reference).

One particularly advantageous approach has been the use of scFv phage-libraries (Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) Nature, 352: 624; Marks et al. (1991) J. Mol. Biol., 222: 581; Chiswell et al. (1992) Trends Biotech., 10: 80; Marks et al. (1992) J. Biol. Chem., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Other systems for generating libraries of polypeptides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection.

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

B. Library Construction.

Libraries for use in selection may be constructed using techniques known in the art, for example as set forth above, or may be purchased from commercial sources. Libraries which are useful in the present invention are described, for example, in WO99/20749. Once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected, as described above, before mutagenesis and additional rounds of selection are performed. Mutagenesis of nucleic acid sequences encoding structurally optimised polypeptides is carried out by standard molecular methods. Of particular use is the polymerase chain reaction, or PCR, (Mulis and Faloona (1987) Methods Enzymol., 155: 335, herein incorporated by reference). PCR, which uses multiple cycles of DNA replication catalysed by a thermostable, DNA-dependent DNA polymerase to amplify the target sequence of interest, is well known in the art. The construction of various antibody libraries has been discussed in Winter et al. (1994) Ann. Rev. Immunology 12, 433-55, and references cited therein.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10X PCR buffer 1 (Perlin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler. The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenized, mismatch is required, at least in the first round of synthesis. The ability to optimise the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

C. Operably Linking One or More Single Light Chain Domains to an Immunoglobulin Scaffold

Preferred methods include the use of polypeptide linkers, as described, for example, in connection with scFv molecules (Bird et al., (1988) Science 242:423-426). Linkers are preferably flexible, allowing the two single domains to interact. The linkers used in diabodies, which are less flexible, may also be employed (Holliger et al., (1993) PNAS (USA) 90:6444-6448).

Domains may be combined using methods other than linkers. For example, the use of disulphide bridges, provided through naturally-occurring or engineered cysteine residues, may be exploited (Reiter et al., (1994) Protein Eng. 7:697-704).

Other techniques for joining domains of immunoglobulins, may be employed as appropriate.

Protein Scaffolds for Use in Constructing Antibody Single Variable Domains and Libraries Thereof

i. Selection of the Main-chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J., 14: 4628; Willams et al. (1996) J. Mol. Biol., 264: 220). Although the H3 region, is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

The immunoglobulins of the present invention are advantageously assembled from libraries of domains, such as libraries of V_{H} domains and libraries of V_{L} domains. Moreover, the single domain ligands of the invention may themselves be provided in the form of libraries. For use in the present invention, libraries of antibody polypeptides are designed in which certain loop lengths and key residues have been chosen to ensure that the main-chain conformation of the members is known. Advantageously, these are real conformations of immunoglobulin superfamily molecules found in nature, to minimise the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use in the invention to assess the number of different main-chain conformations encoded by antibodies, to predict the main-chain conformation based on antibody sequences and to chose residues for diversification which do not affect the canonical structure. It is known that, in the human V_{κ} domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the V_{λ} domain encodes a different range of canonical structures for the L1, L2 and L3 loops and that V_{κ} and V_{λ} domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a preferred aspect, the dual-specific ligands of the invention possess a single known main-chain conformation.

The single main-chain conformation that is chosen is preferably commonplace among molecules of the immunoglobulin superfamily type in question. A conformation is commonplace when a significant number of naturally occurring molecules are observed to adopt it. Accordingly, in a preferred aspect of the invention, the natural occurrence of the different main-chain conformations for each binding loop of an immunoglobulin superfamily molecule are considered separately and then a naturally occurring immunoglobulin superfamily molecule is chosen which possesses the desired combination of main-chain conformations for the different loops. If none is available, the nearest equivalent may be chosen. It is preferable that the desired combination of main-chain conformations for the different loops is created by selecting germline gene segments which encode the desired main-chain conformations. It is more preferable, that the selected germline gene segments are frequently expressed in nature, and most preferable that they are the most frequently expressed of all natural germline gene segments.

In designing single domain ligands or libraries thereof the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1-CS 1 (79% of the expressed repertoire), H2-CS 3 (46%), L1-CS 2 of V_{□}(39%), L2-CS 1 (100%), L3-CS 1 of V_{κ} (36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP47), the J_{H} segment JH4b, the V_{κ} segment O2/O12 (DPK9) and the J_{κ} segment J_{κ}1. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, it is preferable that the chosen conformation is commonplace in naturally occurring antibodies and most preferable that it observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

b. Diversification of the Canonical Sequence

The desired diversity is typically generated by varying the selected molecule at one or more positions. The positions to be changed can be chosen at random or are preferably selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology), 13: 475; Morphosys, WO97/08320).

Since loop randomisation has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

In addition to the removal of non-functional members and the use of a single known main-chain conformation, advantageously only those residues are diversified which are directly involved in creating or modifying the desired function of the molecule. For many molecules, the function will be to bind a target and therefore diversity should be concentrated in the target binding site, while avoiding changing residues which are crucial to the overall packing of the molecule or to maintaining the chosen main-chain conformation.

Diversification of the Canonical Sequence as it Applies to Antibodies

In the case of antibodies, the binding site for the target is most often the epitope binding site. Thus, in a highly preferred aspect, the invention provides libraries of or for the assembly of antibodies in which only those residues in the epitope binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library for use in to the invention. This represents a significant improvement in terms of the functional diversity required to create a range of epitope binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tornlinson et al. (1996) J. Mol. Biol., 256: 813). This complementarity has probably evolved as an efficient strategy for searching sequence space and, although apparently unique to antibodies, it can easily be applied to other polypeptide repertoires according to the invention. According to the invention, the residues which are varied are a subset of those that form the binding site for the target. Different (including overlapping) subsets of residues in the target binding site are diversified at different stages during selection, if desired.

In the case of an antibody repertoire, an initial ‘naive’ repertoire is created where some, but not all, of the residues in the antigen binding site are diversified. As used herein in this context, the term “naive” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens. If required, further diversity can then be introduced outside the region diversified in the initial repertoire. This matured repertoire can be selected for modified function, specificity or affinity.

Useful in the invention are two different naive repertoires of antibodies in which some or all of the residues in the antigen binding site are varied The “primary” library mimics the natural primary repertoire, with diversity restricted to residues at the centre of the antigen binding site that are diverse in the germline V gene segments (germline diversity) or diversified during the recombination process functional diversity). Those residues which are diversified include, but are not limited to, H50, HS52, H52a, H53, HS55, H56, H58, H95, H96, H97, H98, L50, L53, L91, L92, L93, L94 and L96. In the “somatic” library, diversity is restricted to residues that are diversified during the recombination process (junctional diversity) or are highly somatically mutated). Those residues which. are diversified include, but are not limited to: H31, H33, H35, H95, H96, H97, H98, L30, L31, L32, L34 and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries for use in the invention, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favours certain amino acid residues. If the amino acid composition of the ten most diverse positions in each of the V_{H}, V_{κ}, and V_{λ} regions are summed, more than 76% of the side-chain diversity comes from only seven different residues, these being, serine (24%), tyrosine (14%), asparagine (11%), glycine (9%), alanine (7%), aspartate (6%) and threonine (6%). This bias towards hydrophilic residues and small residues which can provide main-chain flexibility probably reflects the evolution of surfaces which are predisposed to binding a wide range of antigens and may help to explain the required promiscuity of antibodies in the primary repertoire.

Since it is preferable to mimic this distribution of amino acids, the invention provides a library wherein the distribution of amino acids at the positions to be varied mimics that seen in the antigen binding site of antibodies. Such bias in the substitution of amino acids that permits selection of certain polypeptides (not just antibody polypeptides) against a range of target antigens is easily applied to any polypeptide repertoire according to the invention. There are various methods for biasing the amino acid distribution at the position to be varied (including the use of tri-nucleotide mutagenesis, see WO97/08320), of which the preferred method, due to ease of synthesis, is the use of conventional degenerate codons. By comparing the amino acid profile encoded by all combinations of degenerate codons (with single, double, triple and quadruple degeneracy in equal ratios at each position) with the natural amino acid use it is possible to calculate the most representative codon. The codons (AGT)(AGC)T, (AGT)(AGC)C and (AGT)(AGC)(CT)—that is, DVT, DVC and DVY, respectively using IUPAC nomenclature—are those closest to the desired amino acid profile: they encode 22% serine and 11% tyrosine, asparagine, glycine, alanine, aspartate, threonine and cysteine. Preferably, therefore, libraries are constructed using either the DVT, DVC or DVY codon at each of the diversified positions.

Sequence Comparisons.

Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is—12 for a gap and —4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Characterisation of the Immunogobulin Molecules of the Invention.

The binding of the immunoglobulin molecules of the invention to specific epitopes can be tested by methods which will be familiar to those skilled in the art and include ELISA. In a preferred embodiment of the invention binding is tested using monoclonal phage ELISA.

Phage ELISA may be performed according to any suitable procedure: an exemplary protocol is set forth below.

Populations of phage produced at each round of selection can be screened for binding by ELISA to the selected epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen, and this can also be undertaken by ELISA using reagents, for example, against a C- or N-terminal tag (see for example Winter et al. (1994) Ann. Rev. Immunology 12, 433-55 and references cited therein).

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

E. Structure of Immunoglobulin Molecules According to the Invention

In the case that the variable domains are selected from V-gene repertoires selected for instance using phage display technology as herein described, then these variable domains comprise a universal framework region, such that is they may be recognised by a specific generic ligand as herein defined. The use of universal frameworks, generic ligands and the like is described in WO99/20749.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair if present

F. (1) Immunoglobulin Molecules of Predetermined Epitope Binding Specificity

According to one aspect of the present invention there is provided an immunoglobulin molecule of predetermined epitope binding specificity comprising one or more single antibody light chain domains which comprises one or more epitope binding specificities operably linked to an immunoglobulin skeleton.

In a preferred embodiment of the above aspect of the present invention, ‘an immunoglobulin skeleton’ is an complete antibody molecule of the IgG subclass lacking at least one light chain variable domain.

Further preferred immunoglobulin skeletons as herein defined include any one or more of those selected from the following: an immunoglobulin molecule comprising (I) the CL (kappa or lambda subclass) domain of an antibody (ii) at least the CH1 domain of an antibody heavy chain, an immunoglobulin molecule comprising the CH1 and CH2 domains of an antibody heavy chain; an immunoglobulin molecule comprising the CH1, CH2 and CH3 domains of an antibody heavy chain; or any of the above subset (ii) in conjunction with the CL domain of an antibody. Those skilled in the art will be aware that this list is not intended to be exhaustive and will be aware of other suitable immunoglobulin skeletons.

Advantageously, an immunoglobulin scaffold' comprises an entire antibody molecule other than the light chain variable domain. Advantageously, the antibody molecule is of the IgG subclass.

(2) Retargeted Antibody Molecules

According to the present invention, the term ‘antibody’ includes within its scope IgG, IgM, IgA, IgD or IgE and fragments such as a FAb, F(Ab′)₂, Fv, disulphide linked Fv, scFv, diabody, dual-specific antibodies whether derived from any species naturally producing an antibody, or created by recombinant DNA technology, whether isolated from serum, B-cells, hybridomas, transfectomas, yeast or bacteria.

In addition, the term ‘antibody’ includes within its scope any fragment of an antibody molecule as herein defined as long as that fragment comprises at least one antigen binding site comprising at least one light chain variable domain and at least one heavy chain variable domain wherein the antigen binding site permits the specific binding of one or more antigens to one or more antibodies of the present invention.

(G) Nucleic Acid Constructs According to the Present Invention.

In general, the nucleic acid molecules and vector constructs required for the performance of the present invention may be constructed and manipulated as set forth in standard laboratory manuals, such as Sambrook et al. (1989) Molecular Cloning: A Laboratory Manual, Cold Spring Harbor, USA.

The manipulation of nucleic acids for use in the present invention is typically carried out in recombinant vectors.

As used herein, vector refers to a discrete element that is used to introduce heterologous DNA into cells for the expression and/or replication thereof. Methods by which to select or construct and, subsequently, use such vectors are well known to one of moderate skill in the art. Numerous vectors are publicly available, including bacterial plasmids, bacteriophage, artificial chromosomes and episomal vectors. Such vectors may be used for simple cloning and mutagenesis; alternatively gene expression vector is employed. A vector of use according to the invention may be selected to accommodate a polypeptide coding sequence of a desired size, typically from 0.25 kilobase (kb) to 40 kb or more in length A suitable host cell is transformed with the vector after in vitro cloning manipulations. Each vector contains various functional components, which generally include a cloning (or “polylinker”) site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media

Since the replication of vectors for use in the present invention is most conveniently performed in E. coli, an E. coli-selectable marker, for example, the β-lactamase gene that confers resistance to the antibiotic ampicillin is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the □-lactamase and lactose promoter systems, alkaline phosphatase, the tryptophan (trp) promoter system and hybrid promoters such as the tac promoter. Promoters for use in bacterial systems will also generally contain a Shine-Delgamo sequence operably linked to the coding sequence.

The preferred vectors are expression vectors that enables the expression of a nucleotide sequence corresponding to a polypeptide library member. Thus, selection with the first and/or second antigen can be performed by separate propagation and expression of a single clone expressing the polypeptide library member or by use of any selection display system. As described above, the preferred selection display system is bacteriophage display. Thus, phage or phagemid vectors may be used. The preferred vectors are phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of a expression cassette that consists (N to C terminal) of a pe1B leader sequence (which directs the expressed polypeptide to the periplasmic space), a multiple cloning site (for cloning the nucleotide version of the library member), optionally, one or more peptide tag (for detection), optionally, one or more TAG stop codon and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southem or Northern analysis, Western blotting, dot blotting of DNA, RNA or protein, in situ hybridisation, immunocytochemistry or sequence analysis of nucleic acid or protein molecules. Those skilled in the art will readily envisage how these methods may be modified, if desired.

H: Use of Immunoglobulin Molecules According to the Invention

Retargeted antibodies and/or antibodies of predetermined epitope binding specificity generated according to the method of the present invention may be employed in in vivo therapeutic and prophylactic applications, in vitro and in vivo diagnostic applications, in vitro assay and reagent applications, and the like. For example antibody molecules may be used in antibody based assay techniques, such as ELISA techniques, according to methods known to those skilled in the art.

As alluded to above, the molecules selected according to the invention are of use in diagnostic, prophylactic and therapeutic procedures. Retargeted antibodies and antibodies of predetermined epitope binding specificity selected according to the invention are of use diagnostically in Western analysis and in situ protein detection by standard immunohistochemical procedures; for use in these applications, the antibodies of a selected repertoire may be labelled in accordance with techniques known to the art. In addition, such antibody polypeptides may be used preparatively in affinity chromatography procedures, when complexed to a chromatographic support, such as a resin. All such techniques are well known to one of skill in the art.

Therapeutic and prophylactic uses of antibodies prepared according to the invention involve the administration of such antibodies generated according to the invention to a recipient mammal, such as a human. A particular advantage of the methods of the present invention is that they can be used to simply and efficiently generate antibodies of the required epitope binding specificity without the need for multiple rounds of selection or complex engineering strategies.

Substantially pure antibodies of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

The retargeted antibodies of the present invention will typically find use in preventing, suppressing or treating inflammatory states, allergic hypersensitivity, cancer, bacterial or viral infection, and autoimmune disorders (which include, but are not limited to, Type I diabetes, multiple sclerosis, rheumatoid arthritis, systemic lupus erythematosus, Crohn's disease and myasthenia gravis).

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the antibodies or binding proteins thereof in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (indstrom et al. (1988) Adv. Immunol., 42: 233). Arthritis is induced in a susceptible strain of mice by injection of Type II collagen (Stuart. et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et aL (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179: 478: and Satoh et al. (1987) J. Immunol., 138: 179).

Generally, the present retargeted antibodies will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The retargeted antibodies of the present invention may be used as separately admininistered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with the retargeted antibodies of the present invention, or even combinations of antibodies according to the present invention having different specificities, such as polypeptides selected using different target antigens, whether or not they are pooled prior to administration.

The route of administration of pharmaceutical compositions or antibodies according to the invention may be any of those commonly known to those of ordinary skill in the art. For therapy, including without limitation immunotherapy, the antibodies, receptors or binding proteins thereof of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of admininistration will depend on the age, sex and condition of the patient, concurrent administration of other drugs, counterindications and other parameters to be taken into account by the clinician.

The antibodies of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present retargeted antibodies or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of antibody per kilogram of body weight, with doses of 0.05 to 2.0 mg/kg/dose being more commonly used. For prophylactic applications, compositions containing the present retargeted polypeptides or cocktails thereof may also be administered in similar or slightly lower dosages.

A composition containing an antibody according to the present invention may be utilised in prophylactic and therapeutic settings to aid in the alteration, inactivation, killing or removal of a select target cell population in a mammal. In addition, the polypeptides described herein may be used extracorporeally or in vitro selectively to kill, deplete or otherwise effectively remove a target cell population from a heterogeneous collection of cells. Blood from a mammal may be combined extracorporeally with the retargeted antibodies, whereby the undesired cells are killed or otherwise removed from the blood for return to the mammal in accordance with standard techniques.

The invention is further described, for the purposes of illustration only, in the following examples.

### EXAMPLE 1.

### Construction of Vectors

a. Construction of the C_{κ} Vector and Ck/gIII Vector.

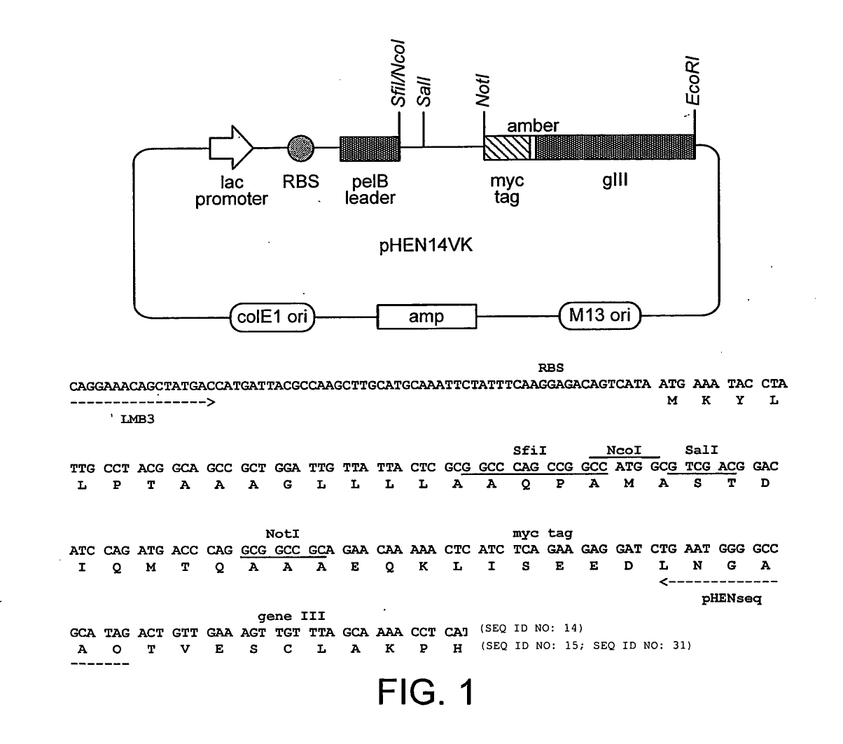

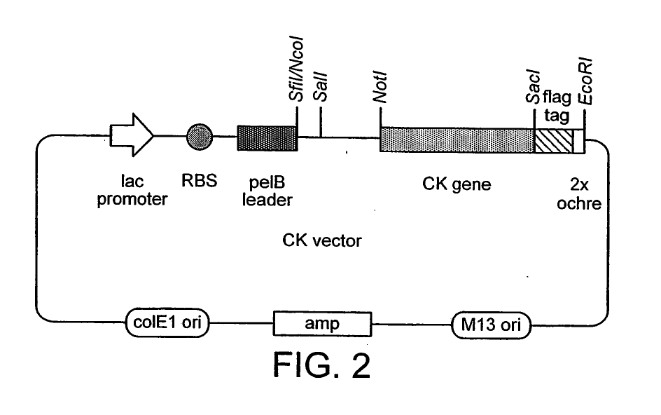

C_{κ} gene was PCR amplified from an individual clone A4 selected from a Fab library (Griffith et al., 1994) using CkBACKNOT as a 5′ (back) primer and CKSACFORFL as a 3′ (forward) primer (Table 1). 30 cycles of PCR amplification were performed as described by Ignatovich et al., (1997), except that Pfu polymerase was used as an enzyme. PCR product was digested with NotI/EcoRI and ligated into a NotI/EcoRI digested vector pHEN14V_{κ} (FIG. 1) to create a C_{κ} vector (FIG. 2).

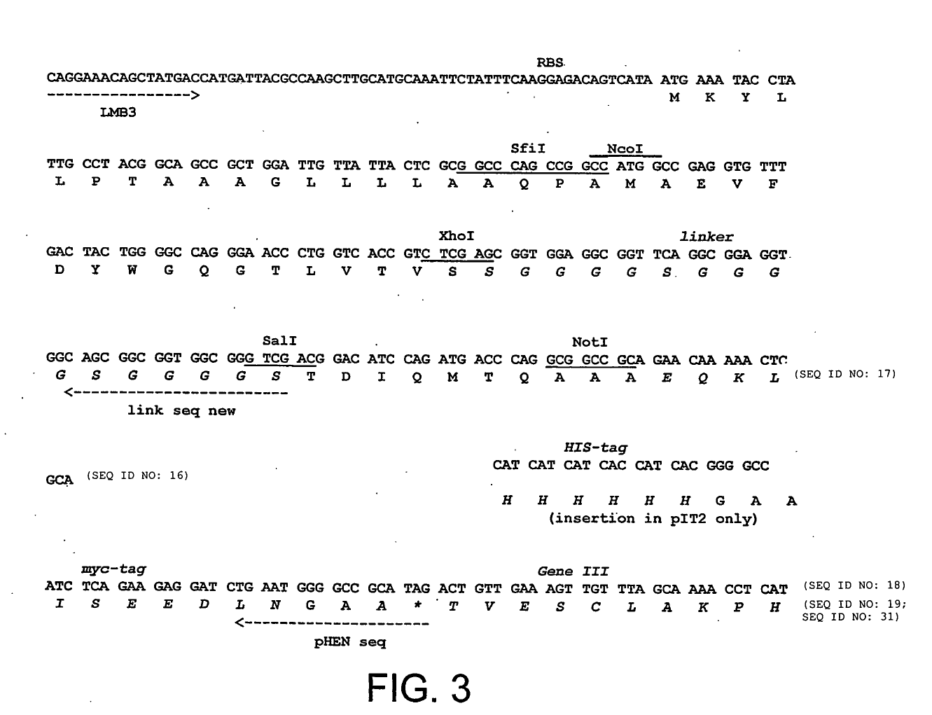

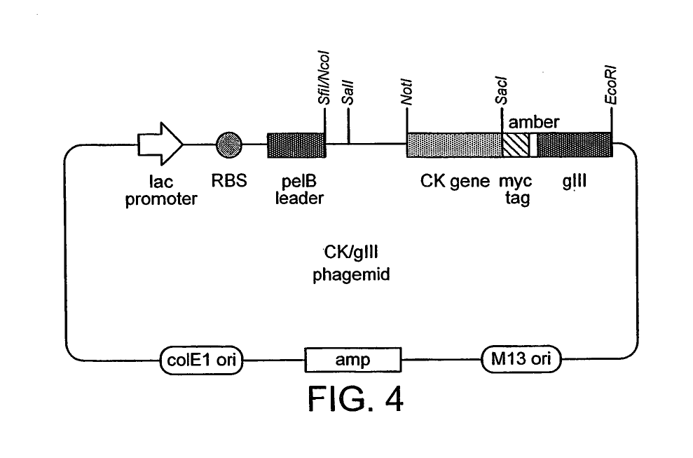

Gene III was then PCR amplified from pIT2 vector (FIG. 3) using G3BACKSAC as a 5′ (back) primer and LMB2 as a 3′ (forward) primer Table 1). 30 cycles of PCR amplification were performed as above. PCR product was digested with SacI/EcoRI and ligated into a SacI/EcoRI digested C_{κ} vector (FIG. 2) to create a Ck/gIII phagemid (FIG. 4).

b. Construction of the C_{H} Vector.

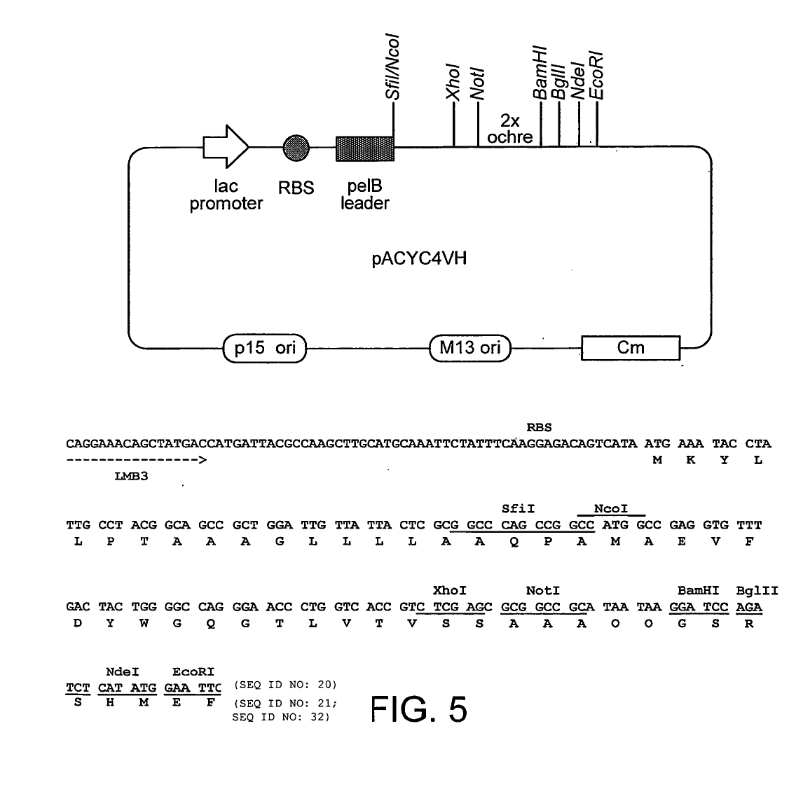

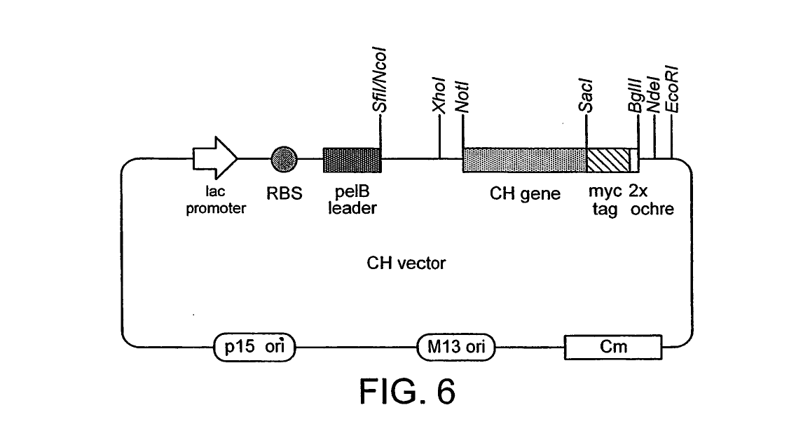

CH gene was PCR amplified from an individual clone A4 selected from a Fab library (Griffith et al., 1994) using CHBACKNOT as a 5′ (back) primer and CHSACFOR as a 3′ (forward) primer (Table 1). 30 cycles of PCR amplification were performed as above. PCR product was digested with NotI/BglII and ligated into a NotI/BglII digested vector PACYC4V_{H} (FIG. 5) to create a C_{H} vector (FIG. 6).

TABLE 1

| | |
|---|---|
| Primer | Sequence (5′→3′) |

| | |
|---|---|
| LMB3 | CAGGAAACAGCTATGAC |
| PHENseq | CTATGCGGCCCCATTCA |
| CkBACKNOT | GCGTCTGCGGCCGCAACTGTGGCTGCACCATCTGTCTTC |
| | ATCTTC |
| CKSACFORFL | ACCAGCCGAATTCTTATTAGCCCTTGTCGTCATCGTCTT |
| | TATAGTCTGAGCTCGCACACTCTCCCCTGTTGAAGC |
| G3BACKSAC | TGTGCGAGCTCAGAACAAAAACTCATCTCAGAAGAGGAT |
| | CTGAATGGGGCCGCATAGACTGTTGAAAGTTGTTTAGCA |
| | AAAC |
| LMB2 | GTAAAACGACGGCCAGT |
| CHBACKNOT | GCGTCTGCGGCCGCCTCCACCAAGGGCCCATCGGTCTTC |
| | C |
| CHSACFOR | GAAGATCTTTATTATGCGGCCCCATTCAGATCCTCTTCT |
| | GAGATGAGTTTTTGTTCTGAGCTCGCACAAGATTTGGGC |
| | TCAACTTTCTTGTCC |
| CKSacHis | CCGGAATTCTTATTAGTGATGGTGATGATGATGTGAGCT |
| | CGCACACTCTCCCCTGTTGAAGC |
| C3MUTFOR | AAGCTCCTGATCTATGAGGCATCCCTTTTGCAA |
| C3MUTBCK | TTGCAAAAGGGATGCCTCATAGATCAGGAGCTT |
| CK.For | AGACTCTCCCCTGTTGAAGCTCTT |
| CH.seq | GGTGCTCTTGGAGGAGGGTGC |

TABLE 2

| | | | BSA ELISA, | |
|---|---|---|---|---|
| | Functionality of | Functionality of | detection with | |
| Clone No | Vk C3HisMut | VH DP-47 | L-HRP | A-HRP |
| 1 | + | + | + | + |
| 2 | + | − | + | − |
| 3 | + | + | + | + |
| 4 | + | + | − | + |
| 5 | + | − | + | − |
| 6 | + | − | + | − |
| 7 | + | + | + | + |
| 8 | + | + | + | + |
| 9 | + | + | − | + |
| 10 | + | − | + | − |

TABLE 3

| | | | BSA ELISA, | |
|---|---|---|---|---|
| | Functionality of | Functionality | detection with | |
| Clone No | Vk C3HisMut | of VH | L-HRP | α-myc |
| 1 | + | DP-2+ | + | + |
| 2 | + | DP-4+ | + | + |
| 3 | + | — | + | − |
| 4 | + | DP-21+ | + | + |
| 5 | + | — | + | − |
| 6 | + | — | + | − |
| 7 | + | DP-4+ | + | + |
| 8 | + | DP-46+ | + | + |
| 9 | + | DP-15+ | + | − |
| 10 | + | DP-10+ | − | − |

### EXAMPLE 2

### Selection of a Single Domain V_{κ}/C_{κ} Antibody (C3) Directed Against BSA from a Repertoire of Single V_{κ}/C_{κ} Antibody Domains Displayed on the Surface of Filamentous Bacteriophage

This example describes a method for making V_{κ}/C_{κ} single domain antibody C3 directed against BSA by selecting a repertoire of virgin V_{κ}/C_{κ} single antibody variable domains displayed on the surface of filamentous bacteriophage for binding to this antigen in the absence of the complementary variable domain.

A repertoire of V_{κ} variable domains was excised from Library 4 SalI/NotI digestion and ligated into a SalI/NotI digested Ck/g3 phagenid (Example 1, FIG. 4) to create a V_{κ}/C_{κ} library (3.6×10⁷) displayed on the surface of filamentous bacteriophage.

Two rounds of selections were performed on BSA using this library. Phage titres went up from 1.2×10³ in the first round to 6.0×10⁷ in the second round. The selections were performed as described previously using immunotubes coated with BSA at 100μg/ml concentration.

After the second round 48 clones were tested for binding to BSA in a soluble V_{κ}/C_{κ} single domain ELISA. 96-well plates were coated with 100μl of 10μg/ml BSA. Production of the soluble V_{κ}/C_{κ} single domain fragments was induced by IPTG as described by Harrison et al., (1996) and the supematant (50μl) containg V_{κ}/C_{κ} single domains assayed directly. Soluble V_{κ}/C_{κ} single domain ELISA was performed as soluble ScFv ELISA described previously and the bound V_{κ}/C_{κ} single domains were detected with Protein L-HRP. 92% of the clones gave ELISA signals above 1.0 (data not shown).

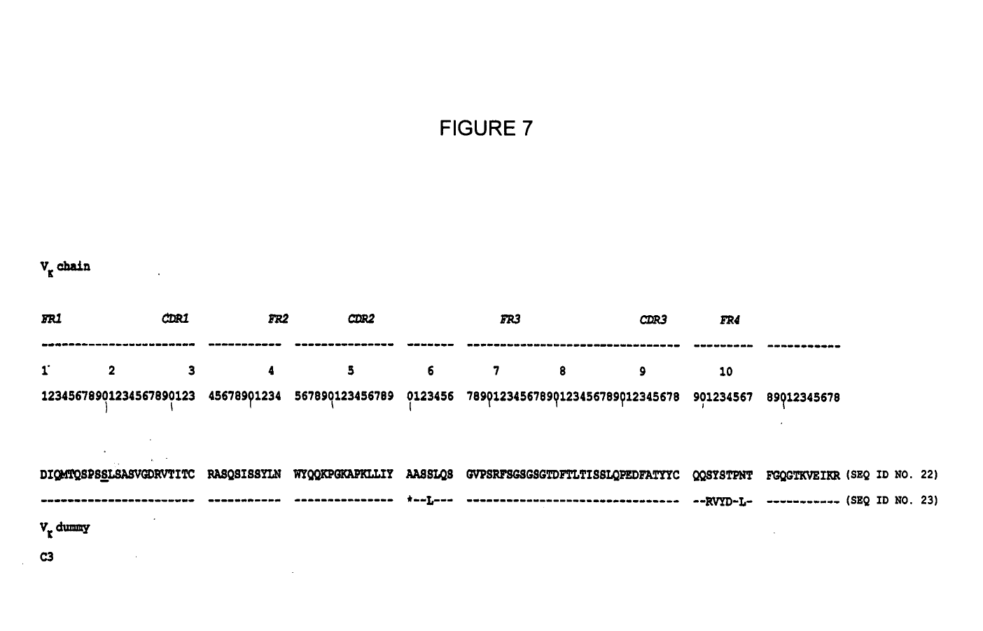

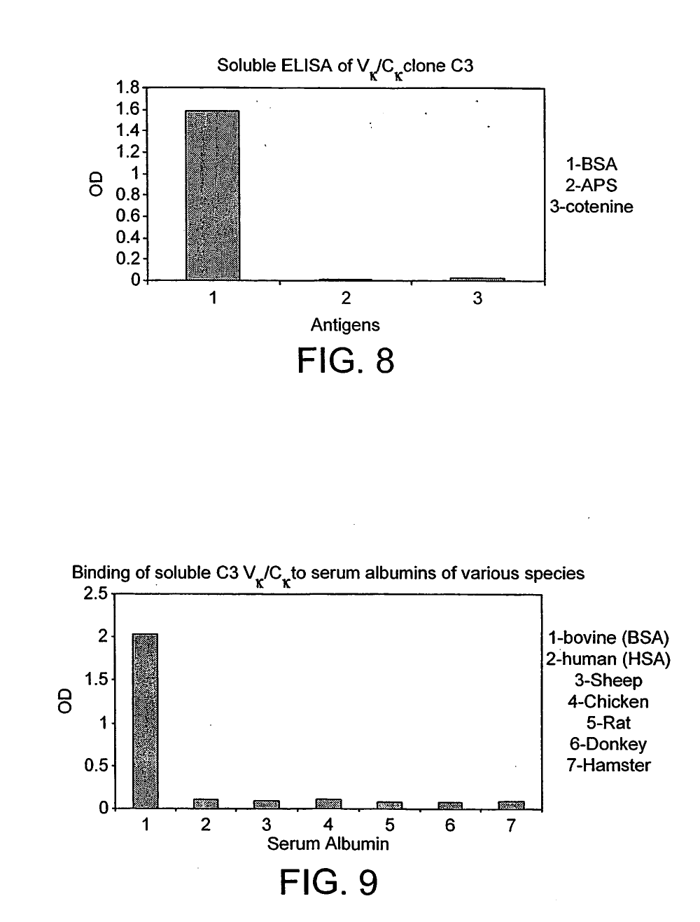

A selection of clones was sequenced as described in Example 1 and one clone (C3) was chosen for further analysis (FIG. 7). Firstly, it was tested for cross-reactivity with other antigens in a soluble V_{κ}/C_{κ} single domain ELISA. 96-well plates were coated with 100 μl of 10μg/ml BSA, APS and cotenine and ELISA was performed as described above. C_{κ}/C_{κ} clone C3 showed specific binding only to BSA (FIG. 8). C3 V_{κ}/C_{κ} was also tested for binding to serum albumins from other species (human, sheep, chicken, rat, donkey and hamster). No cross-reactivity was detected (FIG. 9). C3 V_{κ}/C_{κ} clone was then modified to increase expression levels and facilitate purification of the V_{κ}/C_{κ} variable domain. Firstly, a 6-His tag was put at the 3′ end of the C3 V_{κ}/C_{κ} chain. This was performed by PCR amplification of the DNA from C3 V_{κ}/C_{κ} clone using LMB3 as a 5′ (back) primer and CKSacHis as 3′ (forward) primer (Table 1). 30 cycles of PCR amplification were performed as described in Example 1. PCR product was then digested with SalI/EcoRI and ligated into a SalI/EcoRI digested C_{κ} vector (FIG. 2, Example 1) to create a C3His clone. Secondly, a TAG stop codon in CDR2 of C3 V_{κ} chain (FIG. 3) was changed to GAG (E) according to the protocol of a QuickChange Site Directed Mutagenesis Kit (Stratagene). Two primers C3MUTFOR and C3MUTBCK were used for this purpose (Table 1). DNA was then electroporated into competent HB2151 E. coli cells and the transformants (C3HisMut) were selected on TYE plates containing 1% glucose and 100μg/ml ampicillin. Soluble V_{κ}/C_{κ} fragments from C3HisMut clone were then purified from periplasmic preparation using Protein L-agarose column followed by Ni-NTA column as described by Harrison et al., (1996). 140μg of V_{κ}/C_{κ} C3HisMut chain was obtained from 11 culture (data not shown).

Analysis of the V_{κ}/C_{κ} C3HisMut chain on a denaturing non-reducing protein gel revealed the presence of two bands with molecular weights of 50 kDa and 25 kDa, corresponding to the dimeric and monomeric forms of the protein, respectively (data not shown). The monomer and the dimer were separated by gel filtration using Superdex™ 75 FPLC column and were tested for binding to BSA in ELISA. Only dimeric form of the protein was able to bind BSA (data not shown). The binding affinity of the V_{κ}/C_{κ} C3HisMut dimer was determined by solution phase competition (Friguet et al., 1985) on a BIAcore 2000 biosensor according to Nieba et al., (1996). V_{κ}C_{κ} C3HisMut dimer was found to bind to BSA with a solution affinity of 200 nM.

### EXAMPLE 3

### Creation of Fab Antibody Fragments Containing C3 V_{κ}/C_{κ} C3HisMut Light Chains and Characterisation of their Binding Properties

This example demonstrates that V_{κ}/C_{κ} C3HisMut single domain that is able to bind to BSA in the absence of the complementary variable domain (Example 2) could be combined with a repertoire of virgin complementary V_{H}/C_{H} variable domains to create Fab antibody fragments specific to BSA.

a. Creation of Fab Antibody Fragments Using V_{H} Variable Domains Derived from Library 2 (Example 1, Dual Specific Application).

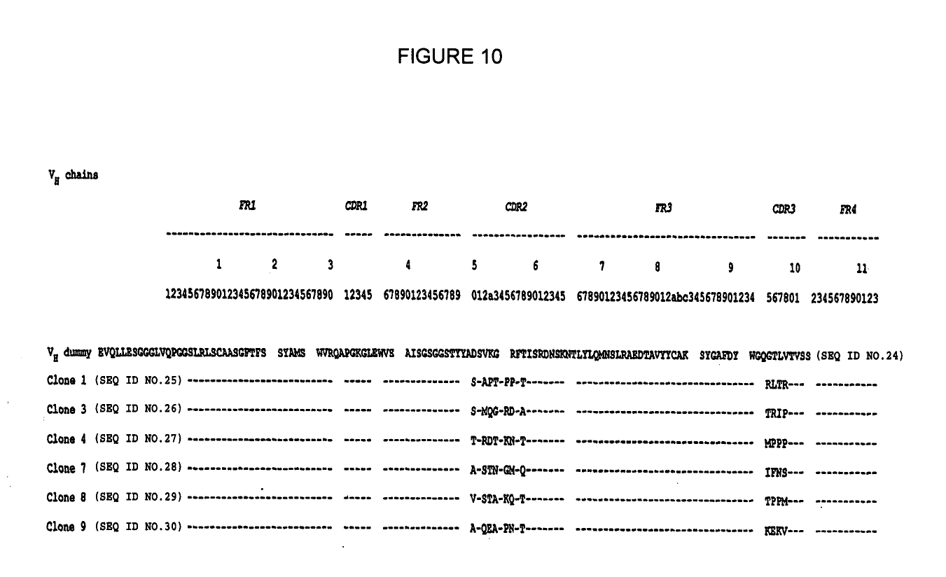

A repertoire of V_{H} variable domains was excised from Library 2 (Example 1, Dual Specific Application) by SfiI/XhoI digestion and ligated into a SfiI/XhoI digested C_{H} vector (FIG. 6) to create a V_{H}/C_{H} library of 2.2×10⁷ clones. DNA prep was then made from this library and used to transform V_{κ}/C_{κ} C3 HisMut clone as described by Chung et al., (1989). Transformants were grown on TYE plates containing 1% glucose, 100μg/ml ampicillin and 10μg/ml chloramphenicol. 10 individual colonies were picked and induced by IPTG to produce soluble Fab fragments. Inductions were performed as described in Example 8 and the supernatant (50μl) containing Fabs assayed directly in ELISA. A 96-well plate was coated with 100μl of 10μg/ml BSA. Soluble Fab ELISA was performed as soluble ScFv ELISA described previously (see Example 1, Dual Specific Application) and bound Fabs were detected with Protein L-BHRP and Protein A-HRP (to check for the presence of light and heavy chains, respectively). V_{H} and V_{κ} variable domains from each of these clones were also PCR amplified and sequenced as described by Ignatovich et al., (1999) using primers LMB3 and Ck.For for V_{κ} and LMB3 and CH.seq for V_{H} variable domain (Table 1). Sequencing results and ELISA results are summarised in Table 2. Out of the clones that produced functional heavy and light chains, 67% were able to bind BSA as Fab fragments. Sequencing revealed that V_{H} variable domains in these clones contained diverse sequences of CDR2 and CDR3 (FIG. 10).

b. Creation of Fab Antibody Fragments Using V_{H} Variable Domains Derived from Griffin ScFv Library.

To check if V_{κ}/C_{κ} C3HisMut single domain (Example 2) could retain its specificity when combined with complementary variable domains of different V_{H} families, a repertoire of V_{H} variable domains was excised from Griffin library by SfiI/XhoI digestion and ligated into a SfiI/XhoI digested C_{H} vector (FIG. 6). Griffin library contains the same synthetic human V-genes as the Human Synthetic Fab 21ox Library (Griffiths, et al., 1994) but is in a ScFv format instead of a Fab format. The total diversity of the Griffin library is 1.2×10⁹ clones.

DNA prep was then made from the created V_{H}/C_{H} library and used to transform V_{κ}/C_{κ} C3HisMut clone as described by Chung et al., (1989). Transformants were grown on TYE plates containing 1% glucose, 100μg/ml ampicillin and 10μg/ml chloramphenicol. 10 individual colonies were picked and induced by IPTG to produce soluble Fab fragments. Inductions were performed as described in Example 8 and the supernatant (50μl) containing Fabs assayed directly in ELISA. A 96-well plate was coated with 100μl of 10μg/ml BSA. Soluble Fab ELISA was performed as soluble ScFv ELISA described in Example 1 and bound Fabs were detected with Protein L-HRP and Anti-c-myc clone 9E10/Anti-mouse-IgG-HRP (to check for the presence of light and heavy chains, respectively). V_{H} and V_{κ} variable domains from each of these clones were also PCR amplified and sequenced as described above. Sequencing results and ELISA results are summarised in Table 3. Out of the clones that produced functional heavy and light chains, 71% were able to bind BSA as Fab fragments. Sequencing of these clones revealed the presence of V_{H} variable domains belonging to different V_{H} families (Table 3).

Thus, the above results indicate that V_{κ}/C_{κ} C3HisMut chain can be combined with a variety of virgin complementary variable domains without loss of its ability to bind BSA.

### EXAMPLE 4

### In vitro Incorporation of the V_{κ}/C_{κ} C3HisMut Chain into a Human Monoclonal IgG Antibody Produced in Mammalian Cells

This example explains a method for creating a BSA binding IgG antibody molecule by combining in vitro a BSA specific V_{κ}/C_{κ} C3HisMut domain (Example 2) with a complementary domain of a complete antibody chain.

A monoclonal IgG antibody 94 of unknown specificity produced in human myeloma cell line (Karpas et al., in preparation) was used in this experiment. The heavy chain of this antibody contains rearranged counterparts of the germline V_{H} gene DP-33 and J_{H}5a and the light chain contains rearranged counterparts of the germline V_{κ} gene DPK9 and J_{κ}2 (data not shown).

125 pmoles of IgG 94 was mixed with 690 pmoles of V_{κ}/C_{κ} C3HisMut (1:5.5 ratio) and incubated with 10 mM DTT at room temperature for 30 minutes to reduce interchain disulphide bonds. The mixture was then dialysed overnight at 4° C. against 1M acetic acid which acted as a denaturing agent and kept heavy and light chains of immunoglobulin apart. The dialysis buffer was then changed to PBS and the mixture was dialysed at 4° C. for 3 days with 3 buffer changes to allow slow reassociation of the heavy and light chains. Since there was an excess of V_{κ}/C_{κ} C3HisMut domain, some V_{κ}/C_{κ} C3HisMut chains should combine with immunoglobulin heavy chains instead of the endogenous light chain. A control experiment with no V_{κ}/C_{κ} C3HisMut added to the IgG 94 was also set up and all stages were carried out as above.

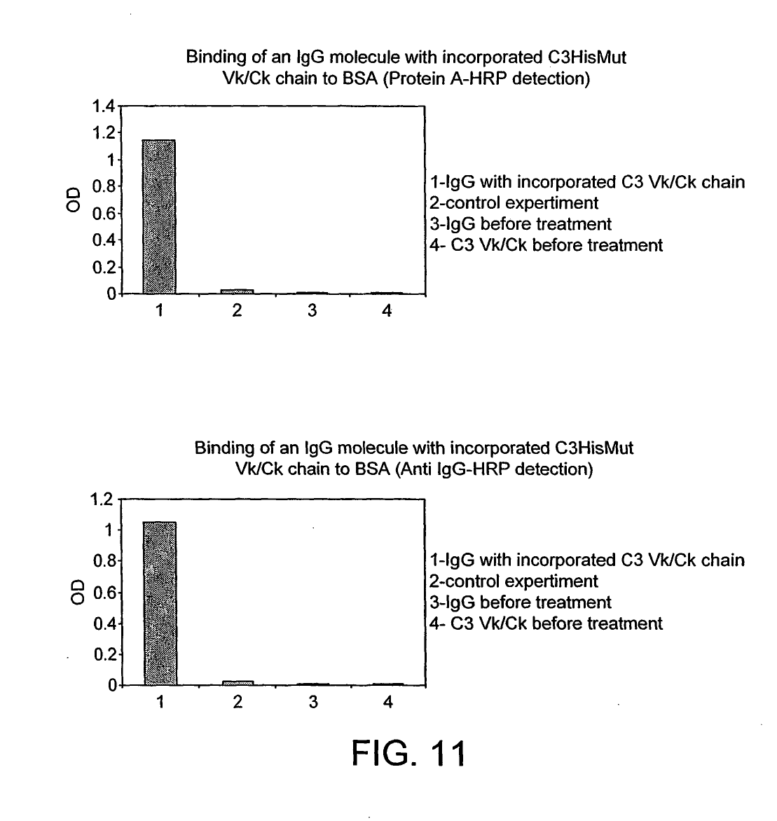

After dialysis the mixtures were analysed for binding to BSA by ELISA. A 96-well plate was coated with 100μl of 10μg/ml BSA. Detection of IgG molecules with incorporated V_{κ}/C_{κ} C3HisMut domain and, therefore, able to bind BSA, was performed with A-HRP and Anti IgG-HRP (Fc specific). ELISA clearly demonstrated that V_{κ}/C₇₈ C3HisMut chains were combined with complementary heavy chains to create an IgG molecule specific to BSA (FIG. 11). Not treated IgG 94 and V_{κ}/C_{κ} C3HisMut chains as well as control experiment gave negative results in this assay (FIG. 11). Moreover, passing the dialysis mixture through Protein A-sepharose column to remove free V_{κ}/C_{κ} C3HisMut chains did not affect ELISA results (data not shown).

### EXAMPLE 5

### In vitro Incorporation of the V_{κ}/C_{κ} C3HisMut Chain Into a Polyclonal IgG Fraction from Human Sera

This example demonstrates that V_{κ}/C_{κ} C3HisMut domain (Example 2) could be combined with a repertoire of complementary variable domains of complete antibody chains to create BSA specific IgG molecules.

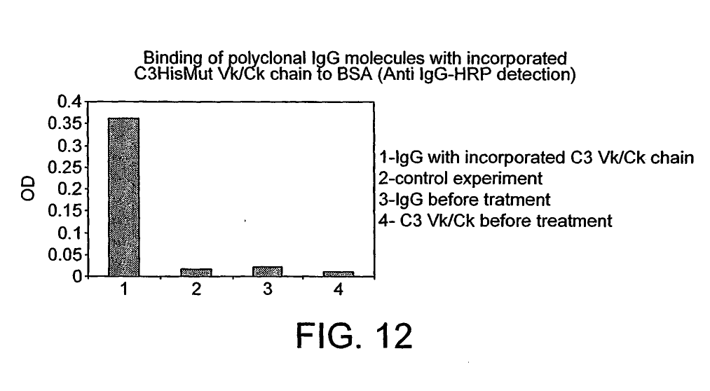

A polyclonal IgG fraction from human sera (Sigma) was used in this experiment to provide a repertoire of heavy chains. Polyclonal IgGs and V_{κ}/C_{κ} C3HisMut chains were treated as described in Example 4. Newly assembled IgG molecules were then tested for binding to BSA by ELISA (Example 4). ELISA demonstrated the presence of IgG molecules that were able to bind BSA (FIG. 12).

All publications mentioned in the above specification, and references cited in said publications, are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for generating an immunoglobulin molecule of predetermined epitope binding specificity comprising the steps of:
(a) selecting an antibody light chain variable domain comprising an epitope binding specificity; and
(b) operably linking the antibody single light chain variable domain to an immunoglobulin skeleton.

2. A method according to claim 1 wherein more than one light chain variable domain is selected according to step (a) and linked to an immunoglobulin skeleton according to step (b), wherein each domain comprises an epitope binding specificity.

3. The method according to claim 1 wherein the immunoglobulin skeleton comprises one or more heavy chain variable domains which together with the one or more antibody light chain domains referred to in step (a) above comprise a specific epitope binding site.

4. A method for retargeting the epitope binding specificity of an antibody molecule such that the resultant antibody molecule is not cross-reactive with the epitope capable of being specifically bound by the antibody molecule prior to retargeting which method comprises the steps of:
(a) selecting an antibody variable domain comprising an epitope binding specificity; and
(b) replacing the light chain variable domain/s or the heavy chain variable domain/s of an epitope binding site comprised by the antibody molecule to be retargeted with the antibody variable domain selected according to step (a), wherein the selected variable domain comprises at least one dominant epitope binding specificity.

5. A method according to claim 4 wherein the antibody variable domain selected according to step (a) comprises an epitope binding specificity wherein the epitope is not structurally related to the epitope specifically bound by the antibody prior to retargeting.

6. A method according to claim 4, wherein the selected variable domain is a heavy chain variable domain.

7. A method according to claim 6 wherein the variable domain replaced according to step (b) is a heavy chain variable domain.

8. A method according to claim 4, wherein the selected variable domain is a light chain variable domain.

9. A method according to claim 8 wherein the variable domain replaced according to step (b) is a light chain variable domain.

10. A method according to claim 8 wherein the selected antibody light chain variable domain is selected from the kappa subgroup of light chain variable domains.

11. An immunoglobulin molecule of predetermined epitope binding specificity obtainable using the method of claim 1.

12. A retargeted antibody molecule obtainable using the method of claim 4.

13. A method for the generation of an antibody of more than one epitope binding specificity comprising the steps of:
(a) selecting an antibody variable domain comprising one or more epitope binding specificities; and
(b) replacing the light chain or the heavy chain variable domain of at least one epitope binding specificity but not all of the epitope binding specificities comprised by the antibody molecule, with the antibody variable domain selected according to step (a).

14. A method according to claim 4 wherein more than one epitope binding site is retargeted.

15. A method according to claim 13, wherein the selected variable domain is a heavy chain variable domain.

16. A method according to claim 13, wherein the selected variable domain is a light chain variable domain.

17. A method according to claim 16 wherein the selected antibody light chain variable domain is selected from the kappa subgroup of light chain variable domains.

18. An antibody obtainable by the method of claim 13.

19. A nucleic acid construct encoding an antibody according to claim 18.

20. A vector comprising a nucleic acid construct according to claim 19.

21. A host cell transfected with a nucleic acid construct according to claim 19.

22. A method for the generation of a dual-specific antibody comprising the step of
(a) selecting an antibody single variable chain domain comprising an epitope binding specificity, wherein at least one epitope binding specificity of the variable domain is co-dominant; and
(b) replacing the light chain variable domain or the heavy chain variable domain of at an epitope binding site comprising the antibody molecule with the antibody variable chain domain selected according to step (a) such that each variable domain of the epitope binding site binds a respective epitope wherein the respective epitopes are not identical to one another.

23. A method according to claim 22 wherein more than one epitope binding site is reformatted as a dual-specific binding site.

24. A method according to claim 22 wherein the two respective epitopes bound by each epitope binding site are not structurally related.

25. A method according to claim 22 wherein the dual-specific antibody has an IgG format.

26. A method according to claim 22 wherein the light chain variable domain of an epitope binding site according to step (b) is replaced.

27. A method according to claim 22 wherein the heavy chain variable domain of an epitope binding site according to step (b) is replaced.

28. A method according to claim 26 or claim 27 wherein the variable domain of an epitope binding site according to step (b) is replaced by a light chain variable domain comprising a second/alternative epitope binding specificity.

29. A method according to claim 26 or claim 27 wherein the variable domain of an epitope binding site according to step (b) is replaced by a heavy chain variable domain comprising a second/alternative epitope binding specificity.

30. A method for retargeting the epitope binding specificity of a polyclonal antiserum comprising the step of:
(a) selecting an antibody variable domain comprising an epitope binding specificity; and
(b) replacing the light chain variable domain or the heavy chain variable domain of at least a proportion of those antibodies comprising the antiserum with the antibody variable chain domain selected according to step (a), wherein the variable domain comprises a dominant epitope binding specificity such that the epitope binding specificity of said portion of antibodies is retargeted.

31. A method according to claim 30 wherein more than one variable domain is selected according to step (a) and used according to step (b) of claim 30.
